(19) United States
(12) Patent Application Publication
(10) Pub. No.: US 2025/0380908 A1
(43) Pub. Date: Dec. 18, 2025

(54) System and Method for Signal Quality Measurement for Digital Biomarkers & Compliance Monitoring

(71) Applicant: Verily Life Sciences LLC, Dallas, TX (US)

(72) Inventors: SUHAS GANESH, San Bruno, CA (US); MILES BENNETT, San Bruno, CA (US); NISHANT VERMA, San Bruno, CA (US)

(21) Appl. No.: 19/181,741

(22) Filed: Apr. 17, 2025

Related U.S. Application Data
(60) Provisional application No. 63/650,458, filed on May 22, 2024.

(51) Int. Cl. A61B 5/00; A61B 5/024; A61B 5/08; A61B 5/1455

(52) U.S. Cl. CPC A61B 5/7221; A61B 5/02405; A61B 5/02416; A61B 5/0816; A61B 5/14551; A61B 5/7264

(57) ABSTRACT

Systems and methods for analyzing a blood volume signal are described. In an example, the method comprises extracting self-normalized features from blood volume signals within a signal window; generating a signal quality prediction based on the self-normalized features to provide a quality prediction score, wherein the quality prediction score is between an upper bound and a lower bound; and generating a health metric score based on the blood volume signals in the signal window if the quality prediction score is above a predetermined threshold. In an example, the blood volume signals are photoplethysmography (PPG) signals. In an example, the method includes identifying a signal quality issue if the quality prediction score is below the predetermined threshold.

### CROSS-REFERENCES TO RELATED APPLICATIONS

This application claims the benefit and priority of U.S. Provisional Application No. 63/650,458, filed on May 22, 2024, the entire disclosure of which is enclosed herein in its entirety.

### TECHNICAL FIELD

This disclosure relates generally to systems and methods for analyzing a blood volume signal, and, in particular, analyzing blood volume signal quality.

### BACKGROUND INFORMATION

Blood volume measurements, such as photoplethysmography (PPG), are sensing modalities commonly used in wearable devices, such as smartwatches, fitness trackers and smart rings, to measure blood volume changes at different parts of the body, such as a finger and a wrist. With each heartbeat, the heart pumps blood through the cardiovascular system and arterial blood volume changes can be measured using PPG, where each cardiac cycle manifests as a peak in the PPG signal. This presents the opportunity to continuously measure physiologically meaningful digital measures, such as heart rate and respiration rate, using PPG signals.

However, PPG signal quality is quite sensitive to a variety of confounding factors, such as motion, loose wear, body sweat, skin tone and other physiological factors. In the presence of such confounders, PPG signal quality can be degraded and can result in inaccurate and unreliable digital measures, such as heart rate. Therefore, it is important to be able to measure PPG signal quality such that, when the signal quality is degraded, digital measures are either not returned to the user/clinician or returned with a confidence score, reflecting the reliability of the measure. The signal quality measure can also be helpful in compliance monitoring, where it can be used to detect when a device is malfunctioning in the field or when a user is not wearing the device as intended.

Measuring PPG signal quality can be a challenging task for a number of reasons. For example, measuring PPG signals reliably can be challenging as it is difficult to a-priori define the exhaustive list of all confounders in real-world usage that will affect PPG signal quality and the type of signal artifacts generated by such confounders. Further, because of this limitation, PPG signal quality often is measured only for specific tasks, such as heart rate, where it is much easier to define the list of signal quality confounders that may impact the task at hand.

Additionally, multiple signal quality metrics, such as signal-to-noise ratio and signal entropy, are sometimes used to measure all aspects of signal quality. Since such signal quality metrics have different units and scale, it can be challenging to interpret all metrics together. In certain instances, methods define a set of signal quality metrics and perform simple boolean operations, such as AND and OR, between the metrics to either label PPG signal as good quality (1) or bad quality (0). A limitation of such methods is that the dynamic range of signal quality is lost and can only return a 0 or 1.

Moreover, it can be difficult to collect annotations or ground truth labels on the quality of PPG signals, especially annotations across different types of real-world signal artifacts. As a result, hand-engineered rules for signal quality are sometimes defined, which again are very task-dependent and fail to generalize across different downstream tasks that may want to use a signal quality score.

### BRIEF DESCRIPTION OF THE DRAWINGS

Non-limiting and non-exhaustive embodiments of the subject matter of the present disclosure are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIG. 1 is a schematic illustration of a system according to an embodiment of the present disclosure.

FIG. 2 is a block diagram of a process according to an embodiment of the present disclosure.

FIG. 3A graphically illustrates a comparison of probability density functions (pdf) of PPG signal entropy for two different PPG measuring device hardware versions using self-normalized PPG signal features, according to embodiments of the present disclosure.

FIG. 3B graphically illustrates a comparison of probability density functions (pdf) of PPG spectral signal-to-noise ratios (SNR) for two different PPG measuring device hardware versions using self-normalized PPG signal features, according to embodiments of the present disclosure.

FIG. 3C graphically illustrates a comparison of probability density functions (pdf) of minimum pulse similarity scores for two different PPG measuring device hardware versions using self-normalized PPG signal features, according to embodiments of the present disclosure.

FIG. 3D graphically illustrates a comparison of probability density functions (pdf) of average pulse similarity scores for two different PPG measuring device hardware versions using self-normalized PPG signal features, according to embodiments of the present disclosure.

FIG. 3E graphically illustrates a comparison of probability density functions (pdf) of spectral kurtosis for two different PPG measuring device hardware versions using self-normalized PPG signal features, according to embodiments of the present disclosure.

### DETAILED DESCRIPTION

Embodiments of a system and a method for analyzing a blood volume signal are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Some portions of the detailed description that follow are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “selecting”, “identifying”, “capturing”, “adjusting”, “analyzing”, “determining”, “estimating”, “generating”, “comparing”, “modifying”, “receiving”, “providing”, “displaying”, “interpolating”, “outputting”, or the like, refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such as information storage, transmission, or display devices.

The algorithms and displays presented herein are not inherently related to any particular computer or other apparatus. Various general-purpose systems can be used with programs in accordance with the teachings herein, or it can prove convenient to construct a more specialized apparatus to perform the required method steps. The required structure for a variety of these systems will appear from the description below. In addition, embodiments of the present disclosure are not described with reference to any particular programming language. It will be appreciated that a variety of programming languages can be used to implement the teachings of the disclosure as described herein.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As described further herein in greater detail, in an aspect, the present disclosure provides a method to measure PPG signal quality, tailored for diverse functionalities of wearable devices and applicable to different anatomical locations of wear, such as wrist and finger. The method provides a component of several PPG based methods to effectively measure digital biomarkers, such as heart rate, heart rate variability, respiration rate and sleep measures. For a small window of PPG signal, it can provide a continuous quality score, such as between 0 to 1, or a quality label (e.g., “good” or “poor” quality, which may be used to determine evaluable regions of PPG signal for downstream digital biomarker measurements. In an embodiment, the method comprises an extension to a two-stage model, where regions of “poor” quality windows are classified into different types of quality issues observed. Understanding the type of quality issue can provide useful information for catching device issues and monitoring participant compliance.

As described further herein, in various embodiments, the present disclosure provides a multi-functional approach for assessing signal quality in blood-volume signals is disclosed, with applications in both digital biomarker development and compliance monitoring. In embodiments, the method comprises extracting self-normalized features from blood volume signals in a signal window and predicting a 14 signal quality score between an upper and lower bounds, unlike just a binary output (“good” vs “bad” quality). In an embodiment, the predicted quality score forms a crucial component for generating digital biomarkers based on blood volume signals. Features implemented are generally self-normalized measures, and hence generalizable across different wearable devices at the same anatomical location. In an example, the blood volume signals are photoplethysmography (PPG) signals. Additionally, movement related features are extracted from movement related signal like Inertial Measurement Unit (IMU) signals. Movement features, along with blood volume features can be used to predict the type of quality issue in the signal window if the signal quality score is below a predefined threshold. This can provide important information regarding device issues and user compliance.

The methods of the present disclosure provide numerous advantages, some of which are discussed immediately below. In an embodiment, the method of the present disclosure provides a quality score, such as between 0 and 1, which offers granularity on the quality of the PPG signal, as compared to a binary quality label (e.g., “good” & “bad”). This also allows for application-specific tunable thresholds to determine quality labels based on application requirements. In embodiments, individual signal quality metrics, such as signal entropy and signal-to-noise ratio, used are all self-normalized measures, and are hence generalizable across wearable devices and anatomical locations. In embodiments, the method is not designed or tuned for a specific application and, hence, is suitable for multiple functionalities of signal quality, i.e., along with producing a quality score, it also predicts the type of quality issue observed. Understanding the type of quality issue provides useful information for catching device issues and monitoring participant compliance. In embodiments, the method of the present disclosure uses and/or is trained on real-world free-living datasets for developing the method, which represent signal artifacts typically observed in real world usage. In embodiments, the datasets include wearable devices collecting PPG and inertial measurement unit (IMU) signals. In embodiments, the datasets include different devices at the same anatomical location, which helps tune model parameters ensuring robustness across wearable devices. In embodiments, human experts have annotated PPG signals to provide detailed quality labels, which are used for training the method.

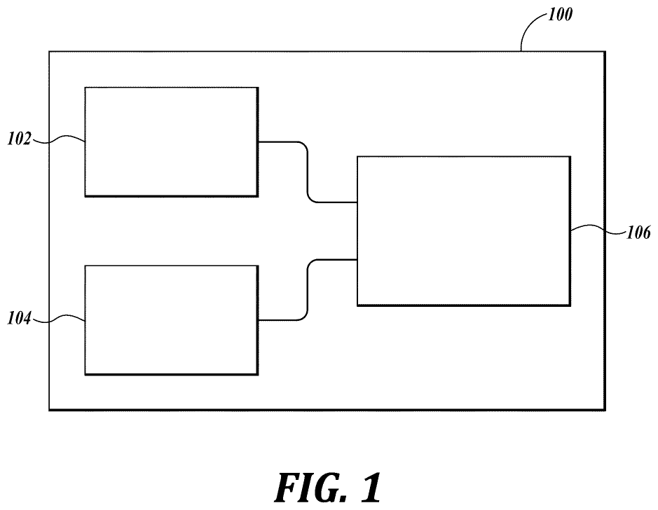

In an aspect, the present disclosure provides a system for measuring and/or analyzing the quality of a blood volume signal. In this regard, attention is directed to FIG. 1 in which a system 100 according to an embodiment of the present disclosure is illustrated.

As shown, the system 100 includes a PPG or other blood volume sensor 102 and a controller 106 operatively coupled to the PPG sensor 102. In an embodiment, the PPG/blood volume sensor 102 comprises a light source, such as a light source configured to emit light into vasculature of a subject, and a photosensor configured to generate a signal based on reflected or transmitted light.

As shown the system 100 further includes a motion sensor 104, such as an inertial sensor 104, configured to generate signals based on movement of the system. As shown, the motion sensor 104 is shown operatively coupled to the controller 106, such as to exchange signals therebetween, such as signals based on movement of the system 100.

In an embodiment, the system 100 is configured to be worn by a subject, such as when generating a PPG or other blood volume signal. In an embodiment, the system 100 is configured to be worn on a portion of the body selected from a wrist, a finger, an car, and the like.

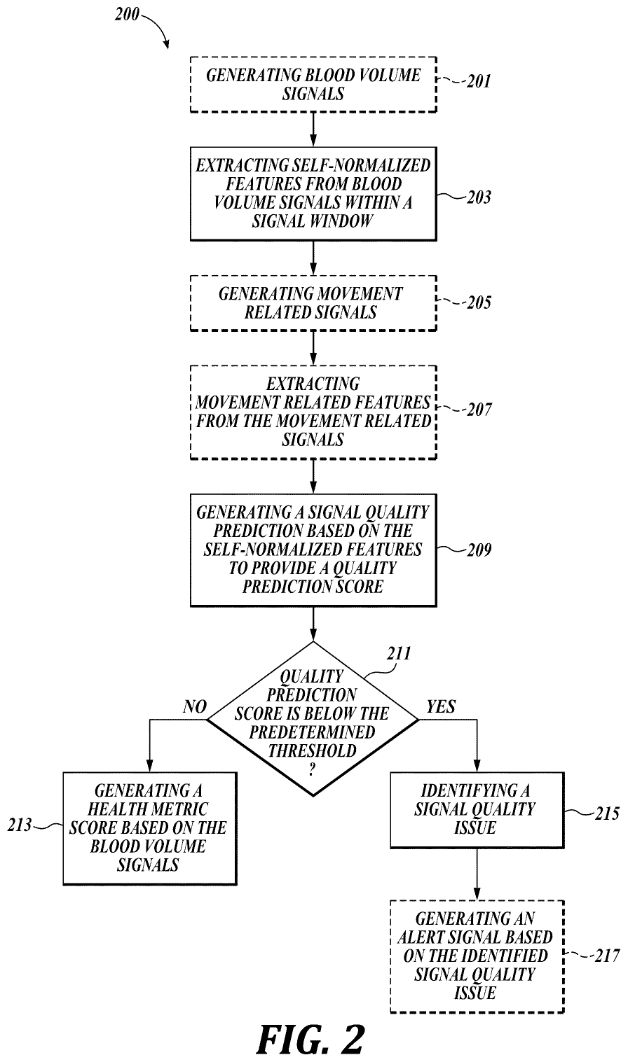

As above, the system 100 includes a controller 106 operatively coupled to various system components, such as the PPG sensor 102 and the motion sensor 104, to choreograph their operation. Controller 106 is coupled with PPG sensor 102 to receive the blood volume signals. Controller 106 is shown further coupled to the motion sensor 104 to receive the movement related signals. Controller 106 can be a computer system (e.g., one or more processors coupled with memory), an application specific integrated circuit (ASIC), a field-programmable gate array, or the like, configured to coordinate and/or control, at least in part, operations of the system 100. Stored on controller 106 (e.g., on the memory coupled with controller 106 or as application specific logic and associated circuitry) are instructions that, when executed by controller 106, perform one or more or a portion of methods of the present disclosure, such as those discussed further herein with respect to FIG. 2.

FIG. 2 is a schematic diagram illustrating an example process 200 for analyzing a blood volume signal. Example process 200 describes a sequence of operations that can be implemented by various hardware elements, including, but not limited to the embodiments of system 100 described further herein with respect to FIG. 1. In particular, a controller (e.g., controller 106 of FIG. 1) can include instructions (e.g., stored on memory) or logic (e.g., an application specific integrated circuit) for performing example process 200. Additionally, or alternatively, example process 200 can be implemented as instructions stored on any form of a non-transitory machine-readable storage medium. In some embodiments, one or more of operations 201-217 of example process 200 can be omitted, repeated, reordered, or executed concurrently (e.g., by parallelization), rather than in sequence as illustrated.

Example process 200 describes a method of analyzing a blood volume signal. In an embodiment, process 200 begins with process block 201, which includes generating blood volume signals. In an embodiment, the blood volumes signals include PPG signals, such as generated with a PPG sensor 102. In an embodiment, the blood volume signals are generated during a signal window, such as during a finite period of time. In an embodiment, the signal window lasts a number of seconds, minutes, hours, days, and the like. In an embodiment, the signal window is in a range of about 1 second to about 1 minute. In an embodiment, the signal window is in a range of about 1 second to about 30 seconds. In an embodiment, the signal window is in a range of about 1 second to about 20 seconds. In an embodiment, process block is optional.

In an embodiment, process block 201 is followed by or method 200 begins with process block 203, which includes extracting self-normalized features from blood volume signals, such as may be generated in process block 201, within a signal window, such as the signal window from process block 201.

In an embodiment, the self-normalized feature is selected from the group consisting of Shannon entropy, sensor saturation, data completeness, spectral signal-to-noise ratio (SNR), spectral kurtosis, pulse morphology similarity, and combinations thereof.

In this regard, the present disclosure uses one or a set of self-normalized features using a machine learning approach, such as a machine learning approach trained on real-world datasets that represent real-world data quality issues. Because the present method uses self-normalized features, it is generalizable across wearable devices and across different anatomical locations. In an embodiment, for different anatomical locations, different real-world datasets are used to retrain the machine learning model because different anatomical locations suffer from different types of data quality issues.

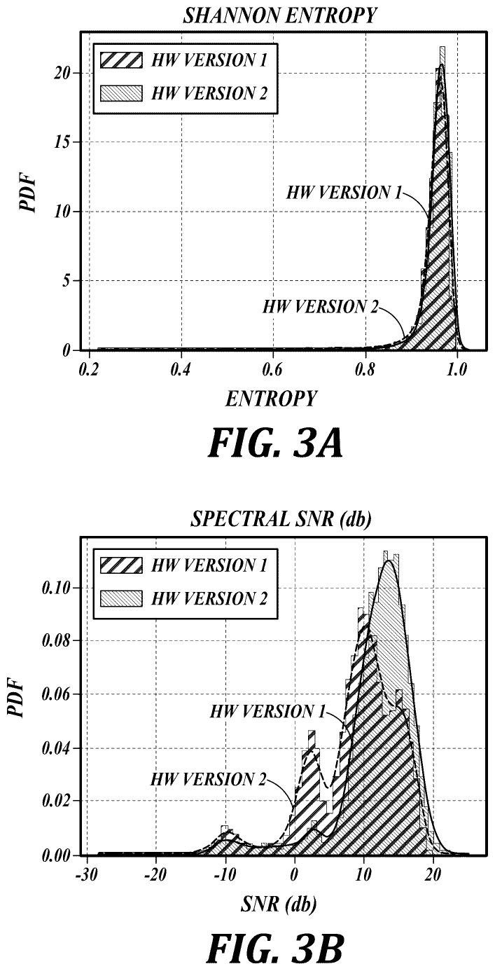

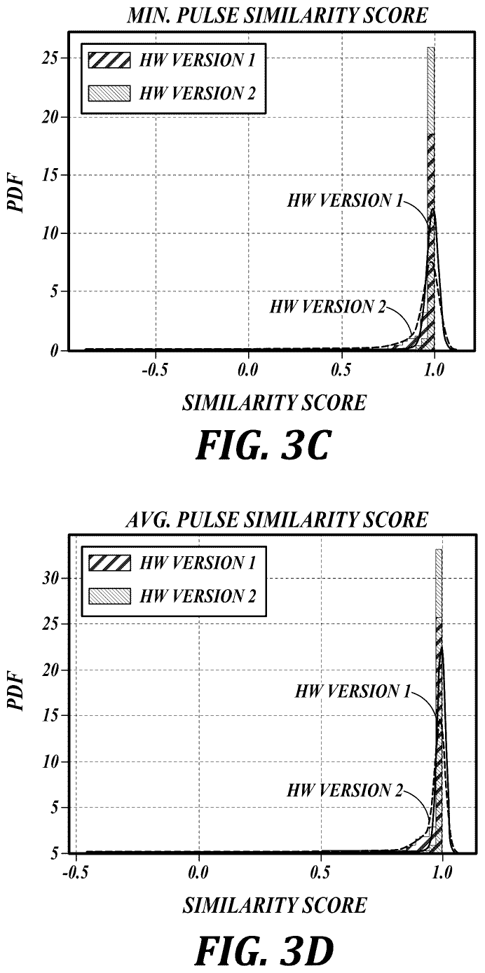

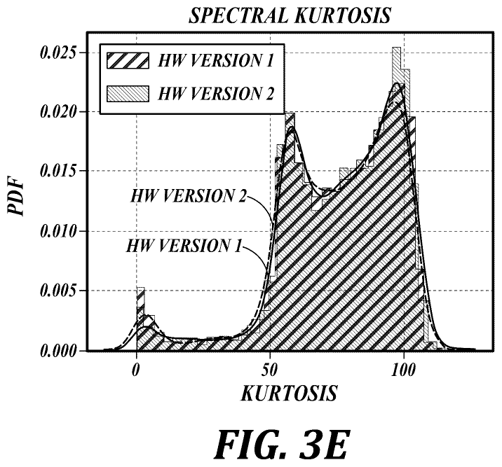

In an embodiment, features implemented are all self-normalized measures, hence generalizable across different wearable devices at the same anatomical location. FIGS. 3A-3E show examples of self-normalized features generalizing across two significantly different versions of a wrist-worn wearable device.

Features are also agnostic to anatomical locations (such as wrist or finger), although the model parameters may be re-trained for different anatomical locations.

In an embodiment, the method uses a self-normalized feature including data completeness. In an embodiment, the signal window segment is analyzed to see if it contains any missing data. If the data is missing, the PPG window may not be used to derive digital biomarkers.

Missing data may be determined in a number of ways. In an embodiment, if a time difference between consecutive data samples crosses a predefined threshold, then the signal window is considered to have missing data and may be rejected or omitted in further analysis. In an embodiment, if there are NaNs or Nulls in the signal, and the duration of continuous NaNs crosses the predefined threshold, then the signal window is considered to have missing data and can be rejected as “bad” quality window or omitted from further analysis.

Missing data may be determined if a data processing pipeline imputes NaNs/nulls in regions where there is missing data, based on expected timestamps.

In an embodiment, a number of valid samples in the signal window is compared with a fraction of expected samples derived from sampling rate to, at least in part, identify missing data in the signal window. Such an approach to identifying missing data in the signal window can identify issues, such as where alternating samples are missing from the data.

In an embodiment, a maximum time difference between consecutive data samples is used as a feature alone, letting the machine learning model learn to predict a quality score.

In an embodiment, sensor saturation, such as a PPG sensor saturation, is extracted from the blood volume signals in the signal window. In an embodiment, the signal window is analyzed to determine whether the signal is corrupted or otherwise defective due to saturation of the sensor. Saturation can happen due to factors such as anatomical and physiological factors, device and sensor-related factors or environmental factors.

Sensor saturation can be determined in a number of ways. In an embodiment, specific saturation flags are stored based on saturation logic defined, for example, on the system. In an embodiment, a sensor datasheet comprises details to track saturation. In an embodiment, rule-based algorithms are configured to identify saturation based on the saturation thresholds.

In an embodiment, the self-normalized feature extracted from the blood volume signals includes Shannon entropy. Shannon Entropy quantifies how much the probability density function (PDF) of the blood volume signal is different from a uniform distribution and, thus, provides a quantitative measure of the uncertainty present in the signal.

In an embodiment, Shannon entropy is calculated as follows:

$$SE = - {\sum }_{i = 1}^{k} \frac{p ( i ) * log ( p ( i ) )}{log ( 1 / k )}$$

where i represents the bin number, and p(i) is the probability distribution of the PPG signal.

Shannon entropy will be 1 if a blood volume signal distribution matches uniform distribution, whereas Shannon entropy will drop to smaller values (<1) when there are motion artifacts, like spikes. In an embodiment, Shannon entropy is used to detect when there are valid pulses in blood volume signal (i.e., high entropy) versus no pulses in PPG signal (i.e., low entropy).

In an embodiment, the presence or absence of a dicrotic notch also produces variability in entropy values. Generally, entropy is lower for fully open/crushed artery cases, which is applicable, for example, for finger-based wearables such as SpO₂ monitor or smart rings. In an embodiment, slight low-frequency modulations make PPG distributions closer to uniform distribution.

In an embodiment, the self-normalized feature extracted from the blood volume signals includes spectral signal-to-noise ratio (SNR). Spectral SNR is a measure of the SNR in the frequency domain. In an embodiment, signal power is estimated by summing powers at an estimated fundamental frequency (f) of heart rate and two other harmonics (2f, 3f). In an embodiment noise power is calculated or otherwise estimated by subtracting signal power from total power of the frequency spectrum. Generally, a good quality blood volume signal comprises clear pulse rate oscillations resulting in high values of SNR.

In an embodiment, a power spectral density is defined as follows:
Pₓ(f)=|X(f)|² where X(f) is the Fourier transform of the signal x(t).

In an embodiment, signal power is defined as follows:
Pₛ=Pₓ(f₀)+Pₓ(2*f₀)+Pₓ(3*f₀) where f₀ is the fundamental frequency.

In an embodiment, total power is defined as follows:

$${P}_{t} = {\sum }_{f = 0}^{N - 1} {P}_{x} ( f )$$

In an embodiment, noise power is defined as follows:

$${P}_{n} = {P}_{t} - {P}_{s}$$

In an embodiment, spectral SNR is defined as follows:

$$SSNR = {P}_{s} / {P}_{n}$$

In an embodiment, computing signal power using a defined signal frequency range (such as heart rate-relevant frequency range of 0.5-4 Hz) is used instead of considering powers at fundamental and harmonic frequencies only.

In an embodiment, computing time-domain based SNR is used instead of spectral based.

In an embodiment, the self-normalized feature extracted from the blood volume signals includes spectral kurtosis. Kurtosis measures the “tailedness” of a probability distribution. Kurtosis describes how much “weight” is in the tails of the distribution compared to the center. Spectral kurtosis analyzes the distribution of power across different frequencies within a signal. High spectral kurtosis indicates the presence of strong transients at specific frequencies, making the spectrum “peaky.” Similar to spectral SNR, a good quality PPG signal with sharp peaks at fundamental frequencies and/or harmonics will have high kurtosis.

In an embodiment,

$$Spectral Kurtosis = E [ {( \frac{{P}_{x} ( f ) - {P}_{a v g}}{{σ}_{{P}_{x}}} )}^{4} ]$$

where E is expectation over all frequencies f and σ_{P}_{ₓ} is standard deviation of the power spectral density Pₓ

In an embodiment, the self-normalized feature extracted from the blood volume signals includes pulse morphology similarity. Signal-level features like Shannon entropy, spectral SNR and kurtosis tend to extract patterns from the entire signal segment, hence capture less effects at individual pulses. Morphology of the PPG pulses could get distorted due to small finger or wrist movements, for example. Hence, it is advantageous to determine the quality of the blood volume signal segment at individual pulse-level. In an embodiment, pulse morphology similarity is extracted by measuring similarity between pulse morphologies in the given blood volume signal window.

In an embodiment, pulse morphology similarity metrics are determined using the following steps. In a first step, pulse detection is performed on the blood volume signals in the signal window, which can be segmented into individual pulses using detected peak and valley locations. In a subsequent step, the amplitude and pulse-width for each segmented pulse is normalized, to remove variability in amplitude and heart rate to influence similarity measures. In a later step, first and last pulses are removed from consideration to account for filtering edge effects and potential incomplete pulses in the signal window. Further, each pulse in the signal window is correlated with every other pulse in the signal window to obtain a pulse correlation matrix. Correlation values for each pulse are averaged to obtain a pulse similarity score, where the pulse similarity score indicates how similar the morphology of the current pulse is with respect to other pulses in the window.

If the similarity score is very low for a pulse and the other pulses in the signal window have high scores, this can indicate that this particular pulse is likely very noisy or distorted.

In an embodiment, average similarity score across all pulses and minimum similarity score are two features, which could be used for later analysis in a machine learning model for predicting signal quality.

Advantageously, normalizing by pulse-width ensures or enhances similarity metrics are robust to variability in heart rate. Further, pulse morphology similarity, as described herein, provides a simpler and effective alternative to template matching based techniques, as it does not require a complex template determination logic. Additionally, a linear correlation measure is robust to small changes in pulse morphologies like changes in pulse transit times, open/closed arteries, presence/absence and shape of dicrotic notches. The pulse morphology analysis methods described herein are also robust to small inaccuracies in determining peak and valley timestamps by the pulse detection algorithm.

In an embodiment, different statistical metrics can be extracted from the similarity scores and used as features, such as mean, median, min, standard deviation, and percentiles, etc. Additionally, instead of performing a linear correlation, an intraclass correlation coefficient (ICC) can be computed if it is advantageous or desired to determine whether pulses are accurately matched. ICC penalizes the correlation value if there are changes in morphological features, such as shape and location of dicrotic notches, and open or closed artery.

In an embodiment, process block 203 is followed by process block 205, which comprises generating movement related signals. In an embodiment, the movement related signals are based on movement of a system, such as system 100, when worn by a subject and, in turn, based on movement of the subject. In an embodiment, the movement related signals are generated by a motion sensor, such as an inertial sensor. In an embodiment, process block 205 is optional.

In an embodiment, process block 203 or process block 205 is/are followed by process block 207, which comprises extracting movement related features from the movement related signals, such as within the signal window. In an embodiment, the movement related signals are correlated, such as correlated in time, with the blood volume signals. In an embodiment, process block 207 is optional.

In an embodiment, motion related signals are extracted to provide additional context to blood volume signals features, such as may be useful to predict the type of quality issue due to which the signal window was denoted as “bad” quality. For example, a noisy or spiky blood volume signal need not necessarily be due to motion artifacts, but when a blood volume signal is coupled with motion related features indicating there was movement in the signal window, this can assist in categorizing the signal window as having “motion artifacts” quality issues.

Motion related signal features can be extracted in a number of ways. In an embodiment, before calculating the motion related features, each axis of an accelerometer signal is represented in standard units of “g”. In an embodiment, statistical metrics, like standard deviation, skewness and kurtosis, can be extracted from each axis (and/or magnitude) of the accelerometer data segment. In an embodiment, these features provide information related to variability, and characteristics of motion or acceleration. In an embodiment, features are averaged across x, y, and z directions to make features invariant to accelerometer axes orientation for a given device.

In an embodiment, correlations between each pair of accelerometer axes can be extracted and included as features in our model. In an embodiment, cross-axis correlation can indicate how strong the synchronization or similarity between the movements captured along different axes are. Further, cross-axis correlation can also help in detecting and differentiating between true movements and noise or artifacts.

In an embodiment, the movement related features are self-normalized.

In an embodiment, process block 207 is optional.

In an embodiment, process blocks 203 or 207 are followed by process block 209, which includes generating a signal quality prediction based on the self-normalized features, such as from process blocks 203 and/or 207, to provide a quality prediction score. In an embodiment, the quality prediction score is between an upper bound and a lower bound, such as continuously between the upper and lower bound. In this regard, the quality prediction score may be anywhere between the upper and lower bound.

In an embodiment, a logistic regression model is trained to predict a quality score between an upper bound and a lower bound, such as between 0 and 1, for each signal window. Such a signal quality score provides a richer and more nuanced assessment of the blood signal quality compared to a simple binary label (i.e., either 1 or zero, or “good” or “bad”).

In an embodiment, logistic regression can be replaced with any other machine learning model, such as a neural network.

As shown, method 200 includes a decision block 211 to determine whether a quality prediction score, such as from process block 209, is below a predetermined threshold. In an embodiment, the predetermined threshold is based on the health metric or other downstream application, and is, thus, variable for particular metrics measured or other downstream applications used, rather than static or fixed irrespective of measured health metric or other downstream application. In an embodiment, a downstream application (such as measuring a health metric or monitoring) might have use cases to use the quality score directly. For example, a sleep versus wake classification algorithm can directly use the quality score as a feature. If not, there's flexibility to choose a specific threshold to determine if the quality is “good” or “bad” based on the application needs.

In an embodiment, decision block 211 is followed by process block 213, which includes generating a health metric score, such as a digital metric, based on the blood volume signals in the signal window if the quality prediction score is above a predetermined threshold. While generating a health metric score, such as a digital metric, based on the blood volume signals in the signal window is discussed herein if the quality prediction score is above a predetermined threshold, it is also possible to generate a health metric score based on the blood volume signals directly, such as without requiring that the quality prediction score is above a particular threshold. In an embodiment, the health metric is selected from the group consisting of heart rate, respiration rate, heart rate variability, a sleep quality measure, and blood oxygen saturation. The health metric score can include a score, assessment, quantification, or other indication of a health metric.

While the terms “above” and “below” are used herein, such as with respect to threshold values, it will be understood that these terms can be reversed depending upon the nature of the signal quality calculation processes.

In an embodiment, decision block 211 is followed by process block 215, which includes identifying a signal quality issue if the quality prediction score is below the predetermined threshold. In an embodiment, the signal quality issue is further based on the movement features. In an embodiment, process block 215 comprises use of a multi-class logistic regression model trained to predict a quality issue for each signal window, when the quality prediction model, such as in process block 209, has labeled the window as “bad” quality (i.e., below the predetermined threshold). In an embodiment, the model learns quality patterns using both blood volume and motion related features from poor quality signal windows to predict probabilities for each quality issue category. In an embodiment, the multi-class logistic regression is replaced with any other multi-class classification model.

Once the signal quality issue is identified, such as in process block 215, method 200 can include process block 217, which includes generating an alert signal based on the identified signal quality issue. Such an alert can be used or otherwise configured to provide a subject, care provider, or other entity with an indication that the signal quality is poor (i.e., below the predetermined threshold) and the nature of the signal quality issue. In the regard, the alert can be used to detect when a device is malfunctioning in the field or a user is not wearing the device as intended.

In an embodiment, the alert can include a message or other indication that, for example, the data in the signal window is of poor quality. In an embodiment, the alert can include a message or other indication that, for example, that the system is being worn by or coupled to the subject in an improper or less preferred manner.

In an embodiment, the alert includes a signal selected from an audible, visible, haptic, or other signal.

As above, the present disclosure provides a two-stage machine-learning model to predict signal quality measures. In an embodiment, the first stage model, such as used in process block 209, predicts the quality of the blood volume segment by predicting a quality score and a quality label. If the window is classified as “bad” quality (i.e., below the predetermined threshold), then the second stage model, such as used in process block 215, predicts the type of the quality issue. The two-stage model simplifies the learning scope for individual models and improves overall performance, especially when the training data is limited.

Developing and training a robust method for blood signal quality measurement is non-trivial, as the space of real-world quality patterns and signal artifacts is large. Therefore, data driven models are advantageous. Certain approaches use simulated datasets or benchtop datasets for training or have limited access to real-world datasets and often lack sufficient signal quality “ground truth” labels.

By contrast, the methods of the present disclosure rely, in certain embodiments, on free-living datasets with wearable devices collecting blood volume and motion related signals, such as of sufficient sample size for training and testing. In an embodiment, human expert annotators have annotated signal windows into “good” or “bad” quality labels. Additionally, if they rated “bad”, they also annotated the type of quality issue for that window. Signal quality methods are trained against these human expert annotations as ground truth. In this regard, the real-world dataset is ensured to have a good representation of different signal artifacts, so that the method learns varied quality patterns.

As discussed further herein, the methods of the present disclosure are generalizable across wearable devices at the same anatomical location. Certain existing approaches use training dataset from a single device. This can result in selecting features that are device specific and not generalizable. The features might not be self-normalized, hence requiring closer attention every time there's a change in hardware version or wearable device.

In embodiments, the methods of the present disclosure extract blood volume and/or motion related features from different wearables devices at the same anatomical location, having different configurations for blood volume and motion sensors. The features selected are self-normalized, which makes such self-normalized features agnostic to device variations. With a training dataset having data from different devices, the model further learns the variability across devices at the same anatomical location, hence improving model generalizability. The trained method is evaluated on an independent dataset with the same device variations to evaluate generalization performance.

For wearable devices at different anatomical locations, the same combination of features and model architecture can be used but, in some embodiments, is re-trained using annotated dataset specific to that location.

The processes explained above are described in terms of computer software and hardware. The techniques described can constitute machine-executable instructions embodied within a tangible or non-transitory machine (e.g., computer) readable storage medium, that when executed by a machine (e.g., controller 107) will cause the machine to perform the operations described. Additionally, the processes can be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A method of analyzing a blood volume signal, the method comprising:
extracting self-normalized features from blood volume signals within a signal window;
generating a signal quality prediction based on the self-normalized features to provide a quality prediction score, wherein the quality prediction score is between an upper bound and a lower bound; and
generating a health metric score based on the blood volume signals in the signal window if the quality prediction score is above a predetermined threshold.

2. The method of claim 1, wherein the blood volume signals are photoplethysmography (PPG) signals.

3. The method of claim 1, wherein the health metric is selected from the group consisting of heart rate, respiration rate, heart rate variability, a sleep quality measure, and blood oxygen saturation.

4. The method of claim 1, wherein the predetermined threshold is based on the health metric.

5. The method of claim 1, wherein the self-normalized feature is selected from the group consisting of Shannon entropy, sensor saturation, data completeness, spectral signal-to-noise ratio (SNR), spectral kurtosis, pulse morphology similarity, and combinations thereof.

6. The method of claim 1, further comprising identifying a signal quality issue if the quality prediction score is below the predetermined threshold.

7. The method of claim 6, wherein the quality signal issue is selected from the group consisting of missing data, motion artifacts, poor SNR, noise artifacts, and combinations thereof.

8. The method of claim 6, further comprising generating an alert signal based on the identified signal quality issue.

9. The method of claim 1, further comprising extracting movement related movement features from movement related signals generated within the signal window.

10. A non-transitory, machine-readable storage medium having instructions stored thereon, which when executed by a processing system, cause the processing system to perform operations comprising:
extracting self-normalized features from blood volume signals within a signal window;
generating a signal quality prediction based on the self-normalized features to provide a quality prediction score, wherein the quality prediction score is between an upper bound and a lower bound; and
generating a health metric score based on the blood volume signals in the signal window if the quality prediction score is above a predetermined threshold.

11. The non-transitory, machine-readable storage medium of claim 10, wherein the blood volume signals are photoplethysmography (PPG) signals.

12. The non-transitory, machine-readable storage medium of claim 10, wherein the health metric is selected from the group consisting of heart rate, respiration rate, heart rate variability, sleep measures, and blood oxygen saturation.

13. The non-transitory, machine-readable storage medium of claim 10, wherein the predetermined threshold is based on the health metric.

14. The non-transitory, machine-readable storage medium of claim 10, wherein the self-normalized feature is selected from the group consisting of Shannon entropy, sensor saturation, data completeness, spectral signal-to-noise ratio (SNR), spectral kurtosis, pulse morphology similarity, and combinations thereof.

15. The non-transitory, machine-readable storage medium of claim 10, wherein the operations further comprise identifying a signal quality issue if the quality prediction score is below the predetermined threshold.

16. The non-transitory, machine-readable storage medium of claim 10, wherein the quality signal issue is selected from the group consisting of missing data, motion artifacts, poor SNR, noise artifacts, and combinations thereof.

17. The non-transitory, machine-readable storage medium of claim 10, wherein the operations further comprise generating an alert signal based on the identified signal quality issue.

18. The non-transitory, machine-readable storage medium of claim 10, wherein the operations further comprise extracting movement related movement features from movement related signals generated within the signal window.

19. A system comprising:
a PPG sensor; and
a controller operatively coupled to the PPG sensor, the controller including logic that, when executed by the controller, causes the system to perform operations comprising:
generating blood volume signals with the PPG sensor;
extracting self-normalized features from blood volume signals within a signal window;
generating a signal quality prediction based on the self-normalized features to provide a quality prediction score, wherein the quality prediction score is between an upper bound and a lower bound; and
generating a health metric score based on the blood volume signals in the signal window if the quality prediction score is above a predetermined threshold.

20. The system of claim 19, further comprising a motion sensor configured to generate motion signals based on movement of the system,
wherein the controller further comprises logic that, when executed by the controller, causes the system to perform operations comprising:
generating the signal quality prediction based on the motion signals.
